(19) United States
(12) Patent Application Publication
(10) Pub. No.: US 2023/0404671 A1
(43) Pub. Date: Dec. 21, 2023

(54) COMPUTER-ASSISTED IMPLANT POSITIONING SYSTEM AND METHODS

(71) Applicant: Corin Limited, Cirencester (GB)

(72) Inventors: Catherine Zoe STAMBOUZOU, Cheltenham (GB); James William PIERREPONT, Cheltenham (GB); Jevan ARULAMPALAM, Cammeray (AU); Moritz Franz EHLKE, Berlin (DE)

(73) Assignee: Corin Limited, Cirencester (GB)

(21) Appl. No.: 17/842,036

(22) Filed: Jun. 16, 2022

(51) Int. Cl. A61B 34/10

(52) U.S. Cl. CPC A61B 34/10; A61B 2034/104; A61B 2034/105; A61B 2034/107

(57) ABSTRACT

A computer-assisted implant positioning system is provided that includes a receiver; a memory operatively connected to the receiver and a processor. The processor is configured to receive preoperative patient-specific data, receive a 3D model of the subject implant, receive an intraoperative patient-specific 2D image of the target bone, register the 3D model of the subject implant to the intraoperative patient-specific 2D image and determine a best fit for the 3D model of the subject implant to the intraoperative patient-specific 2D image, register the 3D bone model of the target bone to the intraoperative patient-specific 2D image of the target bone and determine a best fit for the 3D bone model to the intraoperative patient-specific 2D image, determine an achieved 3D implant position based on the registered 3D model of the subject implant and the registered 3D bone model, and assess the achieved implant position relative to the preoperative patient-specific data.

The present invention relates generally to a computer-assisted implant positioning system and methods for intraoperatively assisting in positioning a surgical implant during a surgical procedure. In particular, the present disclosure relates to a computer-assisted implant positioning system and methods for intraoperatively assisting in positioning a surgical implant using three dimensional (“3D”) computer models of implants and bones aligned with intraoperative images of a patient's bone and implant.

### BACKGROUND OF THE INVENTION

Surgical navigation allows a surgeon to visualize and track surgical instrument positions in three dimensional space. Surgical navigation technology also allows surgeons to track instrument and implant positions relative to a patient's anatomy. Such surgical navigation systems can assist surgeons with, for example, performance of hip, knee, or shoulder joint replacement surgical procedures. In connection with such surgeries, imaging data of a patient can be acquired either preoperatively or intraoperatively, which can assist with the surgical procedure e.g., by enabling the display of various instruments and implants and their positions intraoperatively relative to imaging data of the patient's anatomical structures.

Conventionally the position of an implanted surgical implant during surgery may be verified by eye or palpation intraoperatively by the surgeon, which can result in errors and/or failure to detect misalignment. Intraoperative imaging can also assist a surgeon in performing a qualitative assessment of implant positioning, however this can also result in errors and/or failure to detect misalignment due to the lack of accurate information in three dimensions. Misalignment may result in the failure of the surgical implant or necessitate revision surgery to correct issues arising from undesired misalignment. As such, a need still exists for an apparatus and/or means to better assess three dimensional position and/or alignment of surgical implants during arthroplasty surgery.

### SUMMARY OF THE INVENTION

In accordance with an exemplary embodiment, the subject disclosure provides a computer-assisted implant positioning system comprising: a receiver; a memory operatively connected to the receiver; and a processor operatively coupled to the receiver and the memory, the processor configured to: receive preoperative patient-specific data for a patient that includes: a 3D bone model of a target bone of the patient, and a planned position of a subject implant in the target bone, receive a 3D computer model of the subject implant, receive an intraoperative patient-specific 2D image of the target bone associated with the surgical implant, register the 3D computer model of the subject implant to the intraoperative patient-specific 2D image associated with the surgical implant and determine a best fit for the 3D computer model of the subject implant to the intraoperative patient-specific 2D image associated with the surgical implant, register the 3D bone model of the target bone to the intraoperative patient-specific 2D image of the target bone and determine a best fit for the 3D bone model to the intraoperative patient-specific 2D image, determine an achieved implant position based on the registered 3D computer model of the subject implant and the registered 3D bone model of the target bone, and assess the achieved implant position relative to the preoperative patient-specific data.

In accordance with an aspect of the computer-assisted implant positioning system the processor is further configured to assess the achieved implant position relative to the preoperative patient-specific data to obtain assessment data.

In accordance with an aspect of the computer-assisted implant positioning system the assessment data includes a degree of freedom, orientations, an offset, a lengthening, or combinations thereof.

In accordance with an aspect of the computer-assisted implant positioning system the processor is further configured to output on a display one or more of the assessment data.

In accordance with an aspect of the computer-assisted implant positioning system in registering the 3D computer model of the subject implant to the intraoperative patient-specific 2D image associated with the surgical implant to determine a best fit for the 3D computer model to the intraoperative patient-specific 2D image, the processor is further configured to: acquire an initial position of the 3D computer model of the subject implant; assess the initial position of the 3D computer model of the subject implant to the intraoperative patient-specific 2D image associated with the surgical implant; and refine the initial position of the 3D computer model of the subject implant to match the position of the surgical implant in the intraoperative patient-specific 2D image.

In accordance with an aspect of the computer-assisted implant positioning system in refining the initial position of the 3D computer model of the subject implant to match the position of the surgical implant in the intraoperative patient-specific 2D image, the processor is further configured to match position based on one or more parameters selected from the group consisting of orientation, corners, depth, width, height, size, degree of freedom, and implant anatomical features.

In accordance with an aspect of the computer-assisted implant positioning system in registering the 3D computer model of the subject implant to the intraoperative patient-specific 2D image associated with the surgical implant to determine a best fit for the 3D computer model to the intraoperative patient-specific 2D image, the processor is further configured to: determine the best fit using multi-parameter optimization based on a plurality of position parameters.

In accordance with an aspect of the computer-assisted implant positioning system the plurality of position parameters includes a degree of freedom, orientation, corners, a depth, a width, a height, a size, and/or implant anatomical features.

In accordance with an aspect of the computer-assisted implant positioning system in registering the 3D bone model of the target bone to the intraoperative patient-specific 2D image of the target bone to determine a best fit for the 3D bone model to the intraoperative patient-specific 2D image, the processor is further configured to: acquire an initial position of the 3D bone model of the target bone; assess the initial position of the 3D bone model of the target bone to the intraoperative patient-specific 2D image of the target bone; and refine the initial position of the 3D bone model of the target bone to match the position of the target bone in the intraoperative patient-specific 2D image.

In accordance with an aspect, the computer-assisted implant positioning system further comprising: a patient imaging device operatively in communication with the receiver; and a display operatively connected to the processor.

In accordance with an aspect of the computer-assisted implant positioning system the preoperative patient-specific data includes a surgical plan.

In accordance with an aspect of the computer-assisted implant positioning system the processor is further configured to receive a patient-specific image of a target bone of the patient.

In accordance with an aspect of the computer-assisted implant positioning system the subject implant is a medical implant, a surgical instrument, an implant trial, or a foreign object.

In accordance with an aspect of the computer-assisted implant positioning system the processor is further configured to determine the achieved implant position in 3D relative to target bone.

In accordance with an exemplary embodiment, the subject disclosure provides an implant positioning method comprising: acquiring, using a patient imaging device, an intraoperative patient-specific image of a target bone associated with a surgical implant; acquiring, using a computer, a 3D bone model of the target bone; acquiring, using the computer, a 3D computer model of a subject implant to be implanted in the target bone; registering, using the computer, a position of the 3D computer model of the subject implant relative to a position of the surgical implant in the intraoperative image of the target bone; registering, using the computer, a position of the 3D bone model of the target bone to a position of the target bone in the intraoperative image of the target bone; assessing, using the computer, the position of the registered 3D computer model of the subject implant relative to the position of the registered 3D bone model; evaluating, using the computer, the assessed position of the registered 3D computer models of the subject implant and target bone relative to a planned position of the surgical implant in the target bone of the patient; and outputting on a display, the evaluation of the assessed position of the registered 3D computer models of the subject implant and target bone relative to a planned position of the surgical implant in the target bone of the patient.

In accordance with another aspect of the implant positioning method, wherein the registering step of the 3D computer model of the subject implant comprises: determining, using the computer, an initial position of the 3D computer model of the subject implant relative to a position of the surgical implant in the target bone in the intraoperative patient-specific image; and refining the initial position of the 3D computer model of the subject implant to match the position of the surgical implant in the target bone in the intraoperative patient-specific 2D image.

In accordance with another aspect of the implant positioning method, wherein the step of refining the initial position of the 3D computer model of the subject implant comprises determining a best fit by independently evaluating a plurality of parameters for matching the 3D computer model of the subject implant to the position of the surgical implant in the intraoperative patient-specific 2D image.

In accordance with another aspect of the implant positioning method, wherein the step of refining the initial position of the 3D computer model of the subject implant comprises determining a best fit using multi-parameter optimization to optimize a plurality of parameters for matching the 3D computer model of the subject implant to the position of the surgical implant in the intraoperative patient-specific 2D image.

In accordance with another aspect of the implant positioning method, wherein the registering step of the 3D bone model of the target bone comprises: determining, using the computer, an initial position of the 3D bone model of the target bone relative to a position of the target bone in the intraoperative patient-specific image; and refining the initial position of the 3D bone model of the target bone to match the position of the target bone in the intraoperative patient-specific 2D image.

In accordance with another aspect of the implant positioning method, wherein the step of refining the initial position of the 3D bone model of the target bone comprises determining a best fit by independently evaluating a plurality of parameters for matching the 3D bone model of the target bone to the position of the target bone in the intraoperative patient-specific 2D image.

In accordance with another aspect of the implant positioning method, wherein the step of refining the initial position of the 3D bone model of the target bone comprises determining a best fit using multi-parameter optimization to optimize a plurality of parameters for matching the 3D bone model of the target bone to the position of the target bone in the intraoperative patient-specific 2D image.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the subject disclosure is not limited to the precise arrangements and instrumentalities shown.

In the drawings:

FIG. 1 is perspective view of a computer-assisted implant positioning system in accordance with an exemplary embodiment of the subject disclosure;

FIG. 1A are views of exemplary surgical instruments, implant trials and other foreign objects applicable to the computer-assisted implant positioning system of the subject disclosure;

FIG. 2 is a schematic view of a computer applicable to the computer-assisted implant positioning system of the subject disclosure;

FIG. 2A is a schematic view of a preoperative data module applicable to the computer-assisted implant positioning system of the subject disclosure;

FIG. 3 is an X-ray view of an exemplary planned implant position along with surgical plan data associated with the planned implant position applicable to the computer-assisted implant positioning system of the subject disclosure;

FIG. 4 is an exemplary example of an intraoperative patent-specific 2D X-ray image of a target acetabular and femur bone having a hip cup implant implanted on the acetabulum applicable to the computer-assisted implant positioning system of the subject disclosure;

FIG. 5 is a flowchart of the overall operation of a registration process applicable to the computer-assisted implant positioning system of the subject disclosure;

FIG. 5A is an exemplary flowchart illustrating the overall operation of an assessment of a bone to an implant in accordance with the computer-assisted implant positioning system of the subject disclosure;

FIG. 5B is an exemplary flowchart illustrating the overall operation of an assessment of a bone to another bone in accordance with the computer-assisted implant positioning system of the subject disclosure;

FIG. 5C is an exemplary flowchart illustrating the overall operation of a joint registration of an implant and bone in accordance with the computer-assisted implant positioning system of the subject disclosure;

FIG. 6 illustrates an exemplary initialization of a femur stem implant in accordance with the computer-assisted implant positioning system of the subject disclosure;

FIG. 7 illustrates an exemplary coarse registration of a hip cup implant in accordance with the computer-assisted implant positioning system of the subject disclosure;

FIG. 8 illustrates an exemplary fine registration of a hip cup implant in accordance with the computer-assisted implant positioning system of the subject disclosure;

FIG. 9 illustrates an exemplary coarse registration of a pelvis bone in accordance with the computer-assisted implant positioning system of the subject disclosure;

FIG. 10 illustrates an exemplary fine registration of a pelvis bone in accordance with the computer-assisted implant positioning system of the subject disclosure;

FIG. 11 illustrates an exemplary joint registration of a pelvis bone and hip cup implant in accordance with the computer-assisted implant positioning system of the subject disclosure;

FIG. 12 illustrates an exemplary view of a planned position of a hip implant adjacent a view of an achieved position of a hip implant in accordance with the computer-assisted implant positioning system of the subject disclosure;

FIG. 13A illustrates an exemplary X-ray view of a hip implant overlayed with a femur bone model in accordance with the computer-assisted implant positioning system of the subject disclosure;

FIG. 13B illustrates an exemplary X-ray view of a hip implant (cup and femur stem) overlayed with a silhouette of a femur bone model in accordance with the computer-assisted implant positioning system of the subject disclosure

FIG. 13C illustrates an exemplary X-ray view of a hip implant (cup and femur stem) overlayed with a silhouette of a planned femur stem implant in accordance with the computer-assisted implant positioning system of the subject disclosure;

FIGS. 14A-C illustrate views of an exemplary implant registration in accordance with the computer-assisted implant positioning system of the subject disclosure;

FIGS. 15A-D illustrate views of an exemplary bone registration in accordance with the computer-assisted implant positioning system of the subject disclosure; and

FIG. 16 illustrates an exemplary view of a postoperative image of a target bone and implanted implant in accordance with the subject disclosure.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

The subject disclosure provides for a computer-assisted implant positioning system, as further described below. The computer-assisted implant positioning system includes a receiver, a memory operatively connected to the receiver, and a processor operatively coupled to the receiver and the memory. The processor configured to receive preoperative patient-specific data for a patient that includes a 3D bone model of a target bone of the patient, and a planned position of a subject implant in the target bone. The processor is further configured to receive a 3D computer model of the subject implant, receive an intraoperative patient-specific 2D image of the target bone associated with the surgical implant, register the 3D computer model of the subject implant to the intraoperative patient-specific 2D image associated with the surgical implant and determine a best fit for the 3D computer model of the subject implant to the intraoperative patient-specific 2D image associated with the surgical implant, register the 3D bone model of the target bone to the intraoperative patient-specific 2D image of the target bone and determine a best fit for the 3D bone model to the intraoperative patient-specific 2D image, determine an achieved implant position based on the registered 3D computer model of the subject implant and the registered 3D bone model of the target bone, and assess the achieved implant position relative to the preoperative patient-specific data.

In registering the 3D computer model of the subject implant to the intraoperative patient-specific 2D image associated with the surgical implant, the processor is further configured to acquire an initial position of the 3D computer model of the subject implant, assess the initial position of the 3D computer model of the subject implant to the intraoperative patient-specific 2D image associated with the surgical implant, and refine the initial position of the 3D computer model of the subject implant to match the position of the surgical implant in the intraoperative patient-specific 2D image.

In registering the 3D bone model, the processor is further configured to acquire an initial position of the 3D bone model of the target bone, assess the initial position of the 3D bone model of the target bone to the intraoperative patient-specific 2D image of the target bone, and refine the initial position of the 3D bone model of the target bone to match the position of the target bone in the intraoperative patient-specific 2D image.

The subject disclosure further provides for an implant positioning method. The method comprises the steps of acquiring, using a patient imaging device, an intraoperative patient-specific image of a target bone associated with a surgical implant; acquiring, using a computer, a 3D bone model of the target bone; acquiring, using the computer, a 3D computer model of a subject implant to be implanted in the target bone; registering, using the computer, a position of the 3D computer model of the subject implant relative to a position of the surgical implant in the intraoperative image of the target bone; registering, using the computer, a position of the 3D bone model of the target bone to a position of the target bone in the intraoperative image of the target bone; assessing, using the computer, the position of the registered 3D computer model of the subject implant relative to the position of the registered 3D bone model; evaluating, using the computer, the assessed position of the registered 3D computer models of the subject implant and target bone relative to a planned position of the surgical implant in the target bone of the patient; and outputting on a display, the evaluation of the assessed position of the registered 3D computer models of the subject implant and target bone relative to a planned position of the surgical implant in the target bone of the patient.

In registering the 3D computer model of the subject implant, the method comprises determining, using the computer, an initial position of the 3D computer model of the subject implant relative to a position of the surgical implant in the target bone in the intraoperative patient-specific image; and refining the initial position of the 3D computer model of the subject implant to match the position of the surgical implant in the target bone in the intraoperative patient-specific 2D image.

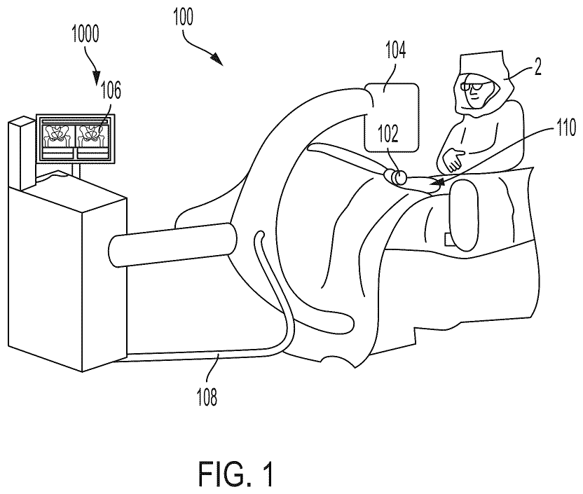

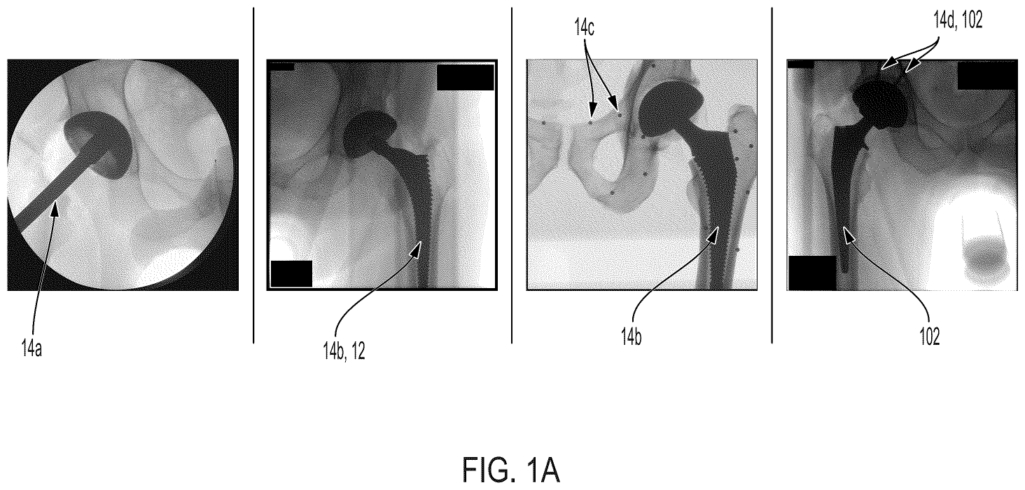

FIG. 1 illustrates a computer-assisted implant positioning system 100 (also referred to herein as “system”) in accordance with an exemplary embodiment of the subject disclosure. The system intraoperatively assists a user 2 (e.g., a surgeon or other medical provider) with positioning a surgical implant 102 to be implanted in a patient during a surgical procedure. This is accomplished by providing the user 2 feedback as to the actual positioning of the surgical implant in the patient relative or in comparison to a planned position of the surgical implant, intraoperatively i.e., before the implant is permanently implanted in the patient. Additionally, the system provides feedback to the user as to the actual positioning of other objects, such as implant trials, surgical instruments, and other objects to tracked during the surgical procedure.

As used herein, the term “position” with respect to positioning a surgical implant, may include an orientation, a depth, an alignment, and/or relative position e.g., relative to bone or other structures, or via x, y, and z coordinates and therefore the position of the implant may be defined using up to six degrees of freedom, including translational and rotational degrees of freedom.

“Patient-specific data” may be any data or data set that may be manipulated or generated that is specific or based on the patient, including patient-specific images, or information derived from such patient-specific images. Such patient-specific data may be used to create or generate 2D images of the patient or 3D images of the patient, and may be obtained preoperatively, intraoperatively, or postoperatively.

“Patient-specific images” are images of the patient e.g., of a patient's bone, such as, but not limited to, X-rays (inclusive of all medical imaging using X-rays such as fluoroscopy), computerized tomography (“CT”) scans/images, magnetic resonance imaging (“MRI”) scans or any other image by known means of imaging the patient. Such patient-specific images may also include intra-operative images of the patient with implants or trial implant instruments implanted in the patient. Images are patient-specific due to the images having a likeness of, being representative of, or identical to the physical bone or anatomical structure of the patient. The patient-specific images or data may also be patient-specific due to an alignment of the subject implant with respect to the patient's bone structure.

As used herein, “intraoperatively” means during the surgical procedure for implanting the surgical implant. Alternatively, “intraoperatively” can mean the time period the patient is under anesthesia for the subject surgical procedure. “Post-operatively” means after the surgical procedure is completed, such as after an implant has been implanted in a patient or after the patient leaves the operating room.

Surgical procedures applicable to the implant positioning system 100 include, but not limited to, orthopedic surgical procedures including arthroplasty. Applicable arthroplasty procedures include e.g., total knee arthroplasty, partial or uni-condylar knee arthroplasty, total hip arthroplasty, ankle joint arthroplasty, elbow joint arthroplasty, shoulder joint arthroplasty, spinal implant procedures, and the like.

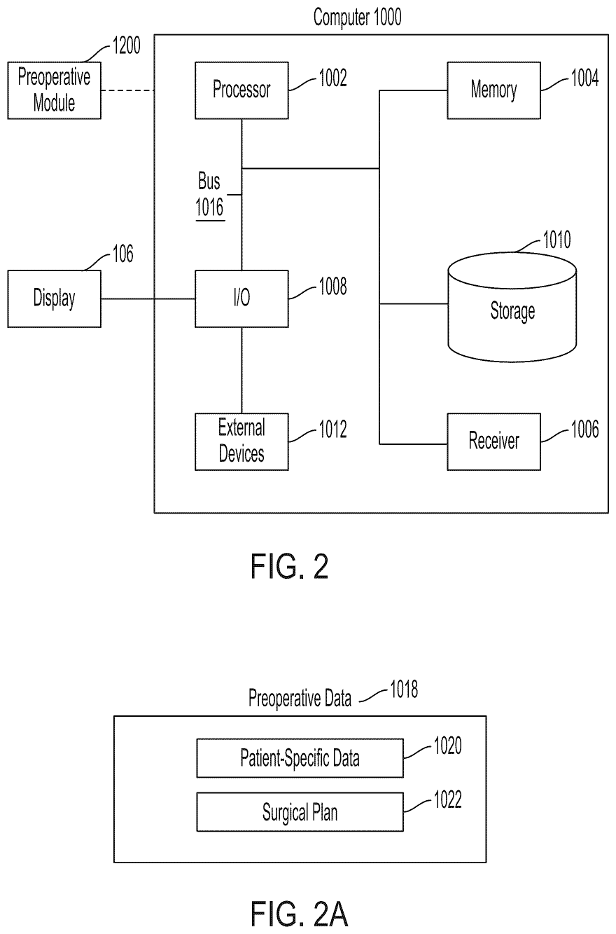

Referring to FIGS. 1 and 2, the computer-assisted implant positioning system 100 includes a computer 1000, a patient imaging device 104 in communication with the computer, and a display 106 in communication with the computer. The computer 1000 includes a processor 1002, a memory 1004, a receiver 1006, Input/Output (I/O) interfaces 1008, a storage 1010, external devices 1012, and a bus 1016 that communicatively couples various system components including the system memory and the receiver to the processor. The processor 1002 is operatively coupled to the receiver.

The computer 1000 typically includes a variety of computer system readable media. Such media may be any available media that is accessible by the computer system, and such media includes both volatile and non-volatile media, removable and non-removable media.

The computer 1000 may also include a program/utility, having a set (at least one) of program modules, which may be stored in the memory 1004 by way of non-limiting example, as well as an operating system, one or more application programs, other program modules, and program data. Each of the operating system, one or more application programs, other program modules, and program data or some combination thereof, may include an implementation of a networking environment. The program modules generally carry out the functions and/or methodologies of embodiments of the subject disclosure as described herein.

The memory 1004 may include computer system readable media in the form of volatile memory, such as random access memory (RAM) and/or cache memory. The computer may further include other removable/non-removable, volatile/non-volatile computer system storage media. By way of example only, the storage system 1010 may be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media may be provided. In such instances, each may be connected to a bus 1016 by one or more data media interfaces. As depicted and described herein in, the memory may include at least one computer program product having a set (e.g., at least one) of program modules that are configured to carry out the functions of the exemplary embodiments of the subject disclosure, e.g., implant positioning.

The receiver 1006 is configured to receive data relating to the patient, such as patient-specific data including implant positioning data. The received data can include preoperative patient-specific data. Preoperative patient-specific data can include patient-specific images e.g., patient X-rays or other images of the patient, preoperative 3D models of a patient's bone, e.g., 3D computer models of a patient's bone generated previously by another system based on patient-specific data, 3D computer models of implants e.g., implants to be implanted or trial implants, applicable to a predetermined plan or surgical plan, a surgical plan which can include an image of a patient having an implant positioned in a pre-determined position such as an ideal implant position along with position information regarding the implant relative to the patient's bone, and the like. The received preoperative patient-specific data may be stored in memory.

In accordance with an exemplary aspect of the present disclosure, the preoperative patient-specific data is generated by a secondary computer system having a Preoperative Module 1200 configured to generate preoperative patient-specific data based on patient-specific data or retrieved from a database of information regarding 3D computer models of implants and instruments and the like.

The bus 1016 represents one or more of any of several types of bus structures, including a memory bus or memory controller, a peripheral bus, an accelerated graphics port, and a processor or local bus using any of a variety of bus architectures. By way of non-limiting example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The computer 1000 may communicate with one or more external devices 1012 such as a keyboard, a pointing device, the display 106, etc.; one or more devices that enable a user to interact with the computer; and/or any devices (e.g., network card, modem, etc.) that enable the computer to communicate with one or more other computing devices. Such communication may occur via the Input/Output (I/O) interfaces 1008. Still yet, the computer system may communicate with one or more networks such as a local area network (LAN), a general wide area network (WAN), and/or a public network (e.g., the Internet) via the receiver 1006. As depicted, the receiver communicates with the other components of the system via the bus 1016. The receiver can also communicate with other local or remote computer systems either e.g., through direct links or remote communication, to receive data relating to implant positioning or any other input data required or accessed by the system.

It should be understood that although not shown, other hardware and/or software components could be used in conjunction with the computer. Non-limiting examples include microcode, device drivers, redundant processing units, external disk drive arrays, Redundant Array of Independent Disk (RAID) systems, tape drives, data archival storage systems, etc.

The surgical implant 102 may be an artificial element that is to be implanted in or on a patient to either replace or be an addition to the patient's joint. Non-limiting examples of a surgical implant may include a hip implant which includes an acetabular cup implant, a femoral stem or femur stem, an acetabular cup liner implant, and a femoral head; a knee implant including femoral, tibial and patellar implants; spinal implants include pedicle screws, bolts, rods, plates, and cages; shoulder implants; elbow implants; ankle implants; and/or other joint and bone implants known in the art or to be developed.

Alternatively, the surgical implant 102 may be a trial implant, e.g., a removable artificial component implanted in or on the patient so as to provide an intraoperative indication of position or size of a final surgical implant. The trial implant can be considered to be a short term or temporary implant and not intended to remain implanted in the patient post-operatively. Other prostheses are also contemplated for the present system 100. That is, any implant or prosthesis may be used with the system that will benefit from verification of position such as alignment, orientation, and/or position relative to the patient's body, such as orthopedic implants including spinal implants.

Various surgical instruments (FIG. 1A) are also used in connection with the computer-assisted implant positioning system. Such surgical instruments can include e.g., a removable artificial component implanted in or on the patient so as to provide an anatomical reference to a bony structure, such as reference markers, pins, bone screws, reference screws, fiducial markers, and/or a pointer for marking positions on a patient's bone.

### Preoperative Data

Preoperative data 1018 (FIG. 2A) received by the system's computer 1000 include preoperative patient-specific data 1020 including data specific to the patient, patient-specific images, 3D bone models of a target bone of a patient (i.e., the patient's bone that is the subject of the surgical procedure) including, 3D computer models of a subject implant (the subject implant being the subject of the surgical procedure, which can be e.g., a medical or surgical implant 102, an implant trial 12, or other foreign object (see FIG. 1A e.g., impactor handle 14a, trial broach 14b, fiducial markers 14c, and screws 14d), a plurality of 3D computer models of an implant family (e.g., 3D computer models of an implant of different sizes), projections of 3D bone models, projections of 3D computer models of a subject implant, a surgical plan, and the like. The target bone can be any bone of the patient, for example, a pelvis, femur, tibia, humerus, scapula, talus, etc. The preoperative data is generated, created or acquired by e.g., the secondary computer system's Preoperative Module 1200 (FIG. 2), and received by the computer 100. The preoperative data can also include feature points of the implant and bone models, as further discussed below.

Exemplary non-limiting 3D bone models applicable to the present disclosure include, the pelvis, femur, tibia, humerus, scapula, talus, etc.

The surgical plan 1022 can include any data related to the subject surgical procedure to assist in achieving an optimal surgical outcome. For example, the surgical plan can include an image of a patient having an implant positioned in a pre-determined position such as an ideal implant position along with position information regarding the implant relative to the patient's bone 4, and the like. The surgical plan can also include patient-specific images, 3D bone models of a target bone of a patient, 3D computer models of a subject implant (the subject implant being the subject of the surgical procedure), a plurality of 3D computer models of an implant family (e.g., 3D computer models of an implant of different sizes), projections of 3D bone models, projections of 3D computer models of a subject implant, images of the position(s) of the subject implant in a target position, target values for implant positioning (e.g., cup inclination and anteversion angles), 3D positions of bone or implant landmarks, biomechanical measures, nominal measurements such as leg length and offset expected as a result of a planned surgery, as well as changes of these measurements from the preoperative to the planned postoperative state.

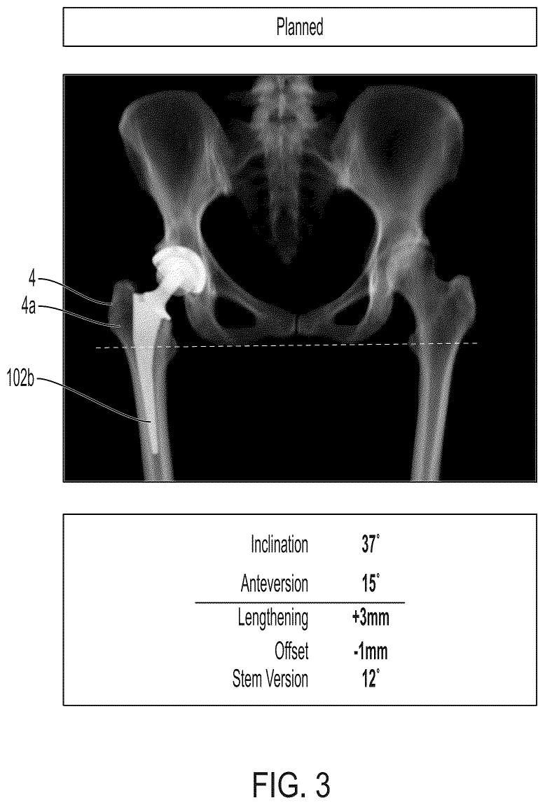

The surgical plan can also include data for a planned, predetermined or ideal position of a subject implant to be positioned on the patient's bone 4, with planned, predetermined or ideal settings for various surgical implant positioning parameters, along with acceptable deviations or range of deviations from the ideal position. In the surgical plan, the 3D bone model can show, have, or indicate a desired relative position and/or orientation of the subject implant with respect to the bone or bone structure of the patient at an ideal position or as determined by a user, which can be defined in 6 degrees of freedom or defined globally relative to a bone. The planned position can be in the form of, but not limited to, a 2D image (referred to as a “planned position image”). FIG. 3 illustrates an exemplary X-ray-like image of a planned position of a hip implant 102b in a hip joint 4a (showing the pelvis and femur) of a patient with planned positioning parameters for inclination, anteversion, lengthening, offset and stem version. The X-ray-like image is an image of a patient's bone virtually implanted with an implant positioned in an ideal implant position, and colored to resemble and X-ray image. The foregoing parameters regarding the surgical plan are herein referred to as “position parameters”.

Alternatively, the system 100 can include a Preoperative Module 1200 similar to the secondary computer system's Preoperative Module. That is, the computer 1000 can additionally include the Preoperative Module for creating, generating or acquiring preoperative data (e.g., via download from a remote source).

Alternatively, the bone structure component of the preoperative patient-specific images may be obtained based on statistical shape modelling, statistical shape analysis, or some other form of inference from a population of patient datasets. The bone structure would thereby be obtained by an averaging, combination, or amalgamation of dimensions of bone structure of multiple persons having similar or identical characteristics of the patient and/or meeting certain criteria. In this way, the bone structure of the 3D bone model may not be identical to the bone structure of the actual patient, and the preoperative patient-specific data may be considered to be patient-specific or patient-class-specific having typical characteristics of the patient. While the foregoing contemplates bone structure components obtained from e.g., statistical shape modeling, it is also contemplated that the bone structure components can be obtained from other means, such as artificial intelligence protocols, machine learning protocols and the like, currently known or to be developed.

A 3D bone model of a target bone is a 3dimensional (3D) computer model of a bone of a patient that is the subject of a surgical procedure. The 3D bone model is created preoperatively by e.g., the secondary computer system's Preoperative Module, and received by the computer 1000. The 3D bone model is stored in memory and used in connection with the bone model registration, as further described below. The 3D bone model may alternatively be a simulation, representation, graphic, drawing, or other type of image of the target bone.

A 3D computer model of a subject implant (alternatively referred to as a “3D implant model”) is a 3-dimensional (3D) computer model of an implant that is the subject of a surgical procedure. The subject implant can be representative of a surgical implant i.e., the implant to be implanted in the patient, a trial implant or temporary implant, or a temporary surgical instrument. The 3D computer model of the subject implant can be created by conventional CAD software e.g., Solidworks or Pro-Engineer and the like. The 3D implant model may alternatively be a simulation, representation, graphic, drawing, or other type of image of the subject implant.

A plurality of 3D computer models of an implant family refers to 3D computer models of the subject implant of various sizes and/or configurations e.g., subject implants of various sizes. A plurality of 3D computer models of an implant family can also refer to 3D computer models of implants that collectively comprise a joint or other collection of implants e.g., a knee joint which includes a femoral implant, a tibial implant, a patellar implant and a tibial insert implant, or a hip joint which includes an acetabular cup implant, a stem implant, a head implant and an acetabular liner implant.

### Computer-Assisted Implant Positioning System

Referring back to FIG. 1, the patient imaging device 104 is communicatively coupled to the computer 1000 e.g., via a data connection 108 and the receiver 1006. The patient imaging device is configured for intraoperatively imaging in two dimensions an operative area 110 of the patient, e.g., for obtaining one or more intraoperative 2D images of the target bone and/or target bone having a surgical implant or implant trial positioned in/on the patient's bone. The intraoperative 2D image of the patient having the surgical implant or implant trial positioned is then used to assess how the implant is positioned relative to the surgical plan i.e., the predetermined ideal position of the implant in/on the patient's bone.

The display 106 is operatively in communication with the computer 1000. The display can be any display known in the art e.g., a computer monitor, screen or visual display.

The patient imaging device 104 may be an x-ray, fluoroscope, ultrasound, CT, MRI, picture archiving and communication system (PACS), Therapy Imaging and Model Management System (TIMMS), or any other patient imaging device known or to be developed suitable for providing patient-specific data or patient-specific image data. This is such that the patient imaging device may directly image the bone structure of the patient and/or the implanted surgical implant 102. The patient imaging device can optionally provide real-time moving or video intraoperative images of the operative area of the patient, particularly in the instance that the patient imaging device is a fluoroscope. The operative area of the patient includes at least part of the bone structure being operated on i.e., the target bone, surrounding bone structure, at least part of the surgical implant being implanted, at least part of the trial implant being implanted, and/or a foreign object e.g., at least part of the surgical instrumentation utilized by the surgeon, but may also include multiple bodies e.g., two bones or two implants.

The patient imaging device 104 may also be configured to output or form an intraoperative 2D image showing objects (e.g., a patient's bone, multiple bones relative to each other, surgical instruments, or surgical implant(s)) at different relative depths to each other. For purposes of the system 100, such images showing objects at different relative depths would be considered to be intraoperative 2D images. Therefore, a patient imaging device that produces a two-dimensional cross-section and a patient imaging device that produces a two-dimensional image showing objects at different relative depths, may be suitable for use by the system. Patient-specific intraoperative 2D images of the target bone and/or the target bone having an implanted surgical implant acquired from the patient imaging device 106 can be displayed on the display 108.

The intraoperative patient-specific 2D image may show a plane or cross-section, or may show objects in a foreground or background. Although the intraoperative patient-specific 2D image is described as showing a real-time position of the surgical implant 102, the position of the surgical implant relative to the bone structure may not necessarily be updated continuously. Therefore, the real-time position or alignment of the surgical implant may refer to an intraoperative position at a given point during the surgery, which could, unintentionally or otherwise, vary between imaging and registration. Such intraoperative patient-specific 2D images may also be of two objects, such as two surgical instruments or two bones of the patient.

The system 100 provides a real-time position for the surgical implant relative to a patient's bone in the intraoperative patient-specific 2D image. While the intraoperative patient-specific 2D image is described as forming a two-dimensional image, the two-dimensional image may not in fact be visible to the user and therefore there may not in fact be an image as such. Instead, the intraoperative patient-specific 2D image may be a data set that can be manipulated to form a two-dimensional image. Additionally, the system can provide real-time positioning of multiple objects e.g., two surgical implants, or multiple bones of the patient.

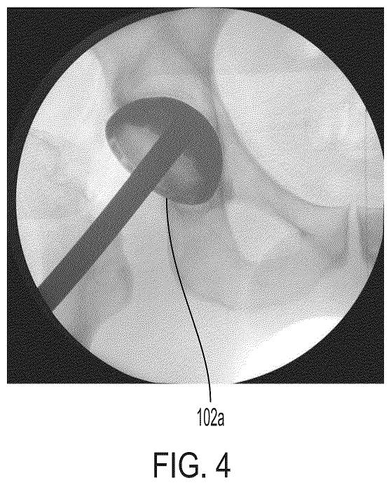

FIG. 4 is an exemplary example of an intraoperative patent-specific 2D image of a target acetabular and femur bone having a hip cup implant 102a implanted on the acetabulum. The image of FIG. 4 is a 2D fluoroscopy image, but the intraoperative patient-specific 2D image can alternatively be any other 2D image by other means, e.g., a plain 2D X-ray radiograph (commonly referred to as “X-rays”).

### Operation

The computer-assisted implant positioning system 100 is a system for performing an arthroplasty procedure. The system includes systems, instruments, and procedures for performing any particular arthroplasty procedure, but a detailed description of such systems, instruments and procedures outside the scope of the present disclosure is not necessary for a complete understanding of the present invention.

After all or all necessary bone cuts and/or bone preparations are made on the patient's bone, trial implants or surgical implants are positioned on/in the bone to allow the user to perform a trial assessment for feel, sizing, range of motion, or any other intraoperative assessment of implant positioning. Once the trial implants or surgical implants are positioned (and before the implants are permanently affixed), the system obtains an intraoperative 2D image of the patient's bone having the implant i.e., an intraoperative 2D image of an achieved implantation (or achieved implantation image) 2000 (see FIG. 4).

The system 100 then performs an assessment of the achieved implantation to determine how well the implant is positioned relative to or in comparison to the patient's anatomy e.g., a target bone, and by extension to the surgical plan, FIG. 12. Alternatively, the system can assess the achieved implantation to any preoperatively targeted position. Additionally, the system can assess the position of the patient's bone relative to another bone e.g., bone to bone position, or the patient's bone relative to a foreign object, e.g., a surgical instrument, or the position of various surgical instruments.

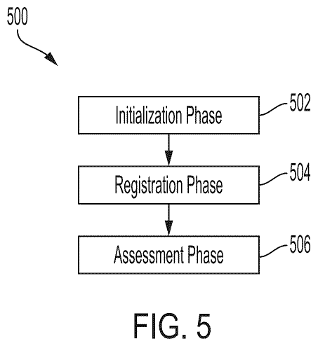

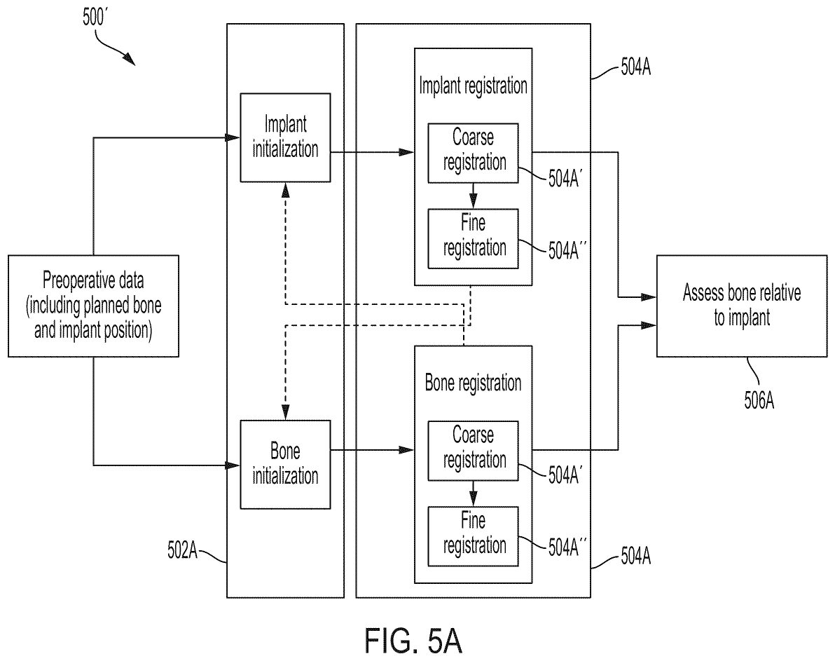

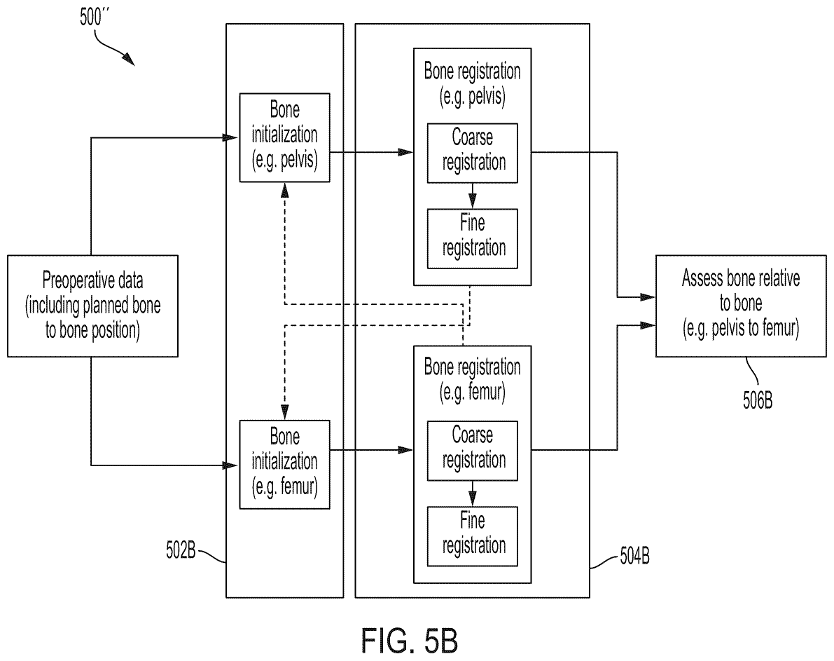

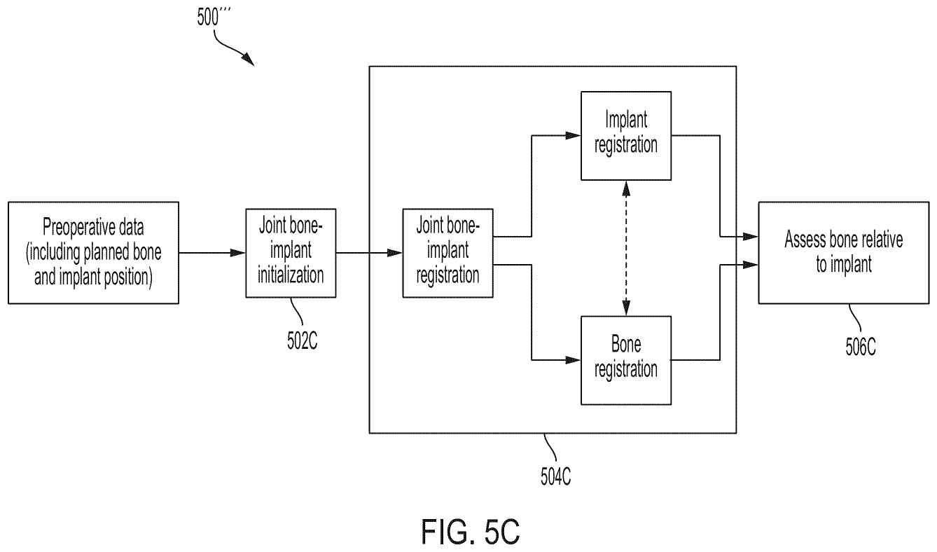

FIG. 5 illustrates a flowchart of the overall operation 500 of the computer-assisted implant positioning system 100. The operation 500 begins with an initialization phase 502, then moves to a registration phase 504, followed by an assessment phase 506. The registration phase 504 includes a coarse registration phase and a fine registration phase. Each phase is conducted intraoperatively with the intraoperative 2D image of an achieved implantation 2000.

The assessment phase 506 can include an assessment of bone to implant (FIG. 5A) or an assessment of bone to bone (FIG. 5B). Referring to FIG. 5A, the operation 500′ includes a bone to implant assessment with preoperative data that can include a planned bone and implant position. The implant and bone are each individually initialized 502A and then registered 504A, and then the bone position is assessed 506A relative to the implant position. Referring to FIG. 5B, the operation 500′ includes bone to bone assessment with preoperative data that can include a planned bone to bone position i.e., a first bone position relative to a second bone position. Each bone e.g., bones of a joint such as a tibia and femur, or pelvis and femur, are each individually initialized 502B and the registered 504B, and then the bone position of the first bone is assessed 506B relative to the second bone.

FIG. 5C illustrates an alternate embodiment of the overall operation 500′″ of the implant positioning system 100. In this exemplary embodiment, the initialization 502C and the registration 504C of the implant model and the bone model is conducted concurrently or jointly, and then the bone registration is used to determine/assess 506C the 3D bone model position relative to the intraoperative 2D image of the achieved implantation, the implant registration is used to determine the 3D implant model position relative to the intraoperative 2D image of the achieved implantation, and/or the relative alignment of bone and implant.

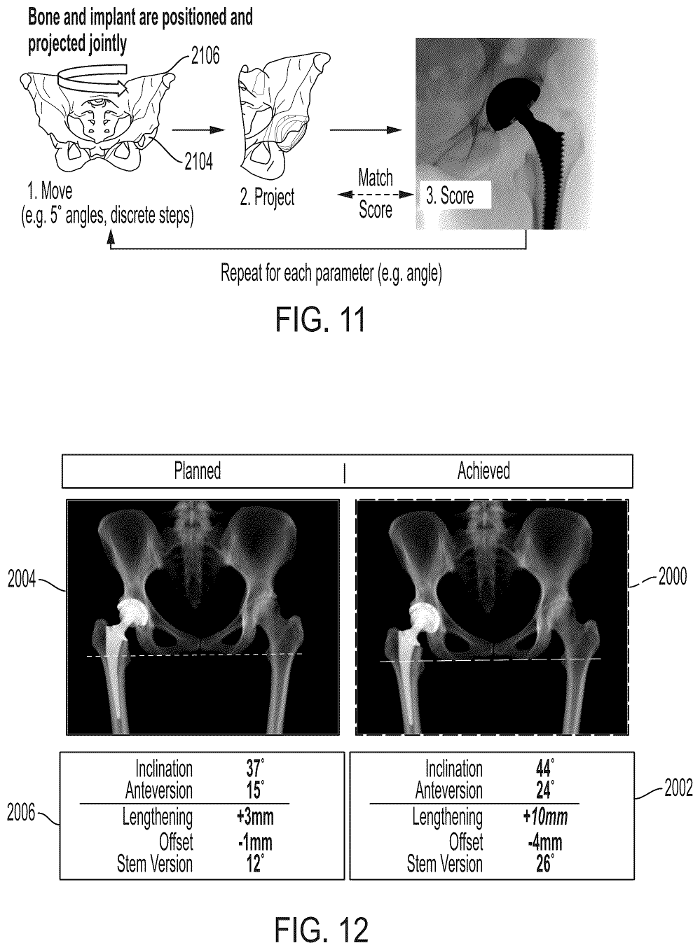

FIG. 11 further illustrates this alternate embodiment of the overall operation 500′″. Benefits of this embodiment is that it uses the position of both the bone and implant to restrict relative movement between them. It also restricts unrealistic positioning between bone and implant and moves both the bone model and implant model simultaneously, thereby reducing potential movement between the bone and implant models as well as the potential for unrealistic positioning between the bone and implant models.

Once the intraoperative 2D image of an achieved implantation is acquired, the operation 500 begins with registering the 3D implant model to the implant in the achieved implantation image, followed by registering the 3D bone model to the patient's bone in the achieved implantation image i.e., the target bone that is the subject of the surgery, and then followed by an assessment of the 3D implant model and 3D bone model relative to the surgical plan e.g., the surgical plan's position of the implant relative to the patient's bone.

### Registration of 3D Implant Model

Registration of the 3D implant model begins with initialization in the initialization phase (see FIG. 5). In the initialization phase, the system determines an initial position of the 3D implant model on the achieved implantation image of the corresponding implant. This is accomplished by determining or selecting a feature point of the implant in the achieved implantation image. The feature point can be e.g., any major feature of the implant, such as a shoulder or shoulder point of a femur stem, an anatomical feature of the implant, or a center, an edge, or surface of the implant in the achieved implantation image. While the foregoing embodiment relates to an implant and 3D implant model, the foregoing invention can be applied to any object e.g., a foreign object to the body, an instrument, and the like, and a 3D model of the object. The system then matches a corresponding feature point of the 3D implant model e.g., the shoulder of the 3D implant model, to the feature point on the achieved implantation image.

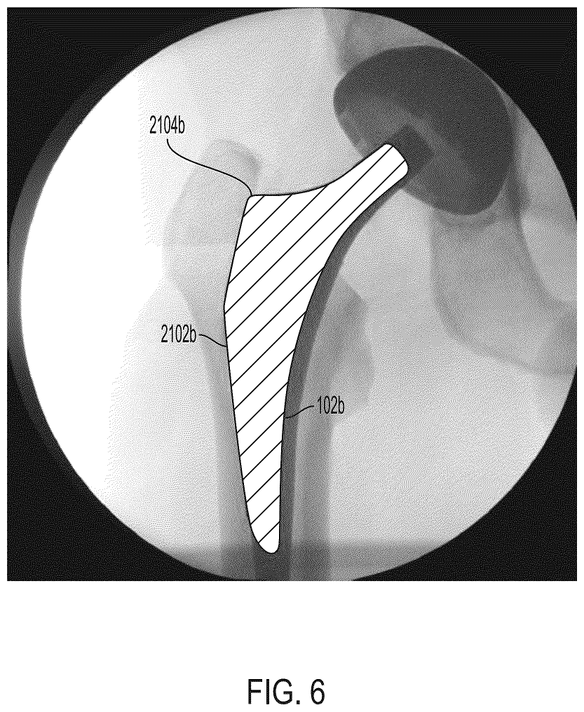

FIG. 6 illustrates an exemplary initialization of a femur stem implant 102b (i.e., a subject implant) in a femur with the feature point of shoulder 2104b of the femur stem of the 3D implant model 2102b being matched to the corresponding shoulder of the femur stem of the achieved implantation image i.e., an intraoperative patient-specific 2D image. This provides an initial match position (i.e., initialization) of the 3D implant model to the corresponding implant on the achieved implantation image.

While the foregoing implant initialization can be accomplished with a single feature point detection, the initial match position can be determined e.g., by aligning a plurality of parameters between the 3D implant model and the corresponding implant of the achieved implantation image. Such parameters can include, but not limited to, position, orientation, translation, 2D image features such as gradient, edges and pixel intensities, 3D model features such as width, height, corner points, any major feature of the implant, an anatomical feature of the implant, a center (e.g., a hip joint center), an edge, or a surface of the implant, a corner, depth, width, height, size, or degree of freedom of the implant.

Alternatively or in addition to, the implant initialization can be conducted by using or applying a registration from a prior image as the initialization for a current image. That is, the implant initialization can be achieved by positioning a 3D model according to the positioning parameters obtained from an earlier registration, e.g. from a registration that was performed on a preceding image in the surgical workflow. This allows to transfer positioning information between successively acquired 2D achieved implantation images.

In accordance with an alternative aspect of the subject disclosure, the preoperative data associated with the initialization phase can include a selection of initial positions (including orientation(s)) and/or feature points of a subject implant based on the subject surgical procedure.

After initialization of the subject implant, the operation moves to the registration phase of the 3D implant model to the implant in the achieved implantation image (i.e., an intraoperative patient-specific 2D image) to determine a best fit of the 3D implant model to the implant in the achieved implantation image. The registration phase 504 A includes a coarse registration phase 504A′ and a fine registration phase 504A″ (FIG. 5A).

Referring to FIG. 5A, the implant positioning system performs a first registration phase 504A′ (also referred to as “coarse registration” or “coarse” optimization”), applicable to the implant registration. During coarse registration of the implant, the system 100 independently tests different values for each position parameter to best match the implant position as depicted in the achieved implantation image. This is accomplished by the system iteratively determining or generating a 2D projection image 2104a of the 3D implant model 2104 and assessing the position parameters of the 2D projection image compared to the position parameters of the implant in the achieved implantation image.

The assessment includes determining a match score based on the 2D information (e.g., position parameters) depicted in the 2D projection image compared to the information (e.g., position parameters) depicted in the achieved implantation image. The match score is then compared to a predetermined score or an ideal score for determining when the iterative process of the coarse registration is considered to be completed, e.g., when the match score reaches the ideal score.

Projections of 3D bone models or 3D computer models of the subject implant are 2D images of the 3D bone models or 3D computer models of the subject implant at a specific position. The 2D projections of 3D models can be generated intraoperatively during the surgical procedure.

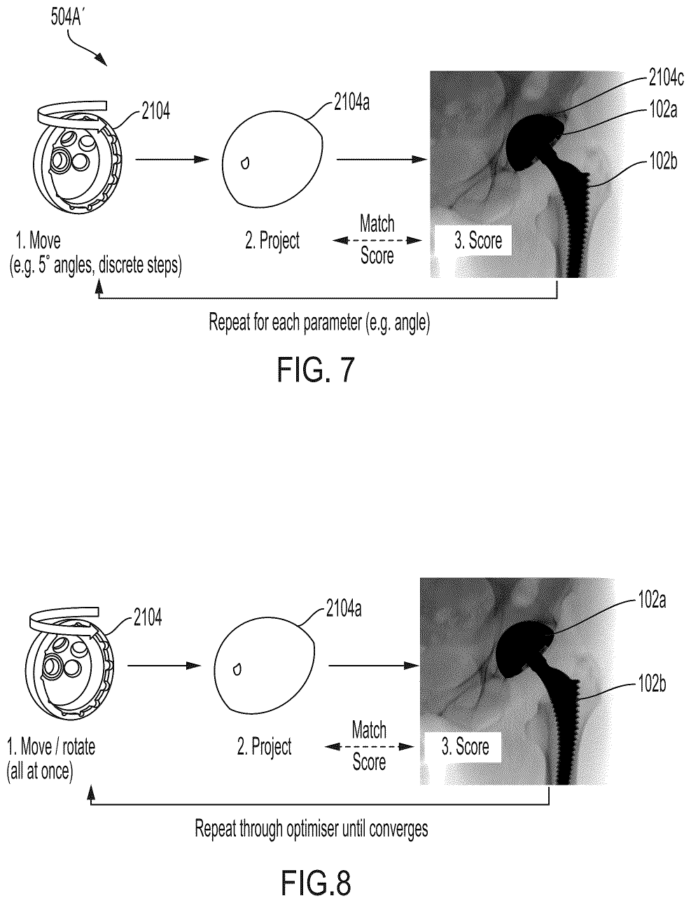

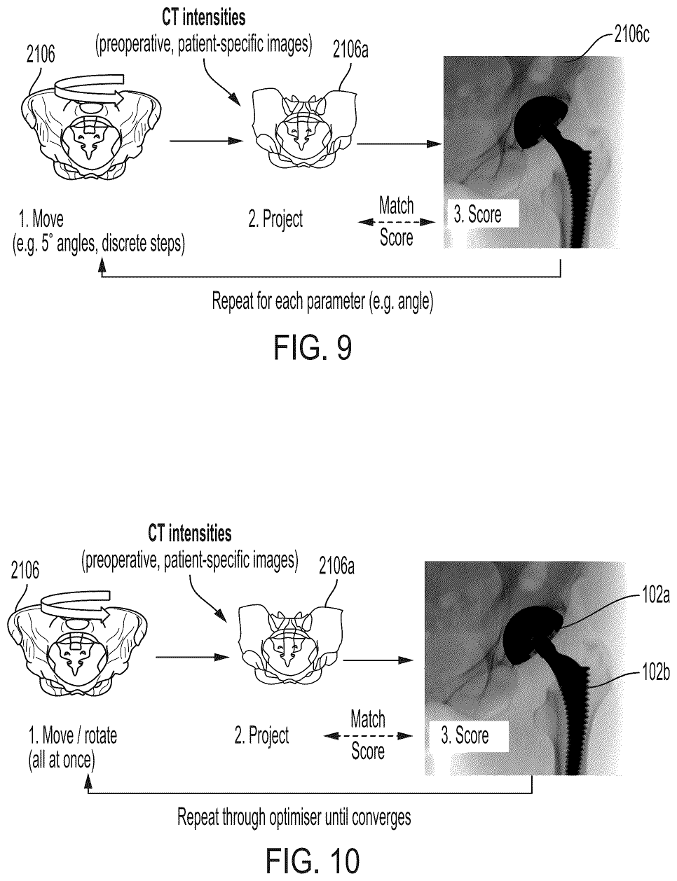

2D projection images are generated by projecting the 3D model information and/or associated 3D points and features (such as 3D landmarks) into the 2D plane, given the position parameters of the model. The 2D projection images can be or depict the contours or silhouettes of the 3D model including landmark points as well as other features of the 3D model. Additionally, the projection images can mimic clinical 2D X-ray or fluoroscopy images given the position parameters of the 3D model. These “virtual X-ray like projections”, also known as “Digitally Reconstructed Radiographs” (DRRs), are computed by tracing rays through the model and accumulating the X-ray attenuation that is encountered by each ray passing through the 3D model (see FIG. 7, virtual X-ray of implant 2104a; FIG. 9, virtual X-ray of bone 2106a).

The match score is defined via a similarity in appearance of the implant model depicted in the 2D projection image and the implant depicted in the achieved implantation image 2104c. The match score is computed based on the distance of corresponding features (e.g., silhouettes, landmarks) between the 2D projection image and the achieved implantation image, and/or by comparing pixel intensity values in the 2D projection to pixel intensity values in the achieved implantation image through measures known in the art, such as, mutual information or gradient correlation.

The position parameters can include feature points as discussed above and/or relative position and orientation, depth position, translation, axis of the implant, angles covering six degrees of freedom, offset, size, 2D image features such as gradients, edges, and pixel intensities, 3D model features such as width, height, and corner points, a center (e.g., a hip joint center) and other anatomical landmarks and the like.

This process defines a coarse registration cycle because each position parameter can be modified in discrete increments e.g., increments of 5 degrees for angles, sizes in increments of 10% of overall size, and relative position in 1 mm increments. Alternatively, the discrete increments can be more or less, such as 4 or 6 degrees, sizes in increments of 5% or 15% or, relative position in 0.5 mm or 2 mm increments and the like. The iterative process ends when each parameter has been evaluated and an optimized match score or overall best match score is achieved.

Upon completion of the coarse registration, the registration phase moves to the second registration phase (sometimes referred to as “fine registration” or “fine optimization”) (FIG. 8). During the second registration phase, the system refines the positions of the 3D computer model of the subject implant relative to the implant in the achieved implantation image. That is, the system refines the position determined during the first phase as the best match position. The best match position determined during the first phase is used as the initial position for the second registration phase, which then determines a best overall match or best overall score (sometimes referred to as best overall match score) for aligning the 3D implant model to the implant in the achieved implantation image. The best overall match or best overall score is a predetermined score, or a score determined by the system based on data from the multi-parameter optimization, as further discussed below.

Referring to FIG. 8, the second registration phase performs similar operations as the steps in the first phase but does so using multi-parameter optimization which performs a continuous search across all position parameters concurrently until a best fit position is determined for all parameters evaluated. That is, the system uses multi-parameter optimization to identify a best fit among one or more of the position parameters. The system can use e.g., Powell optimization (sometimes referred to as Powell's method or Powell's conjugate direction method) to identify a local minimum among the multitude of position parameters scored for determining a best fit position. Alternatively, other multi-parameter optimization techniques know in the art or to be developed and applicable to the present disclosure can also be used to optimize the second registration phase and determine the best fit position. Upon completion of the second registration phase, the 3D implant model is a registered 3D implant model.

### Registration of 3D Bone Model

After the 3D implant model registration, the system registers the 3D bone model 2106 to the bone in the achieved implantation image. The registration of the 3D bone model is performed substantially the same as the registration of the 3D implant model described above. While the 3D bone model registration is described herein to be completed after the 3D implant model registration, the 3D bone model registration can alternatively be completed before the registration of the 3D implant model or simultaneously with the registration of the 3D implant model. Additionally, both the 3D implant model 2104 and 3D bone model 2106 can be registered jointly. FIG. 11 illustrates registration of the 3D implant model and 3D bone model jointly or simultaneously.

Registration of the 3D bone model begins with initialization in the initialization phase (FIG. 9). In the initialization phase, the system determines an initial position of the 3D bone model 2106 on the achieved implantation image of the corresponding implant. This is accomplished by determining or selecting a feature point of the bone in the achieved implantation image. The feature point can be e.g., any major or anatomical feature of the bone, axis of bone, or a center, an edge, or surface of the bone in the achieved implantation image. Alternatively, the initial position can be achieved based on the known position of the bone or implant from a previous registration, and/or via preoperative planning information such as the planned relative alignment between bone and implant (e.g., planned pelvis position relative to the cup), the anatomical pose of the patient from preoperative imaging (e.g., pelvic tilt, femur abduction/adduction), and the like.

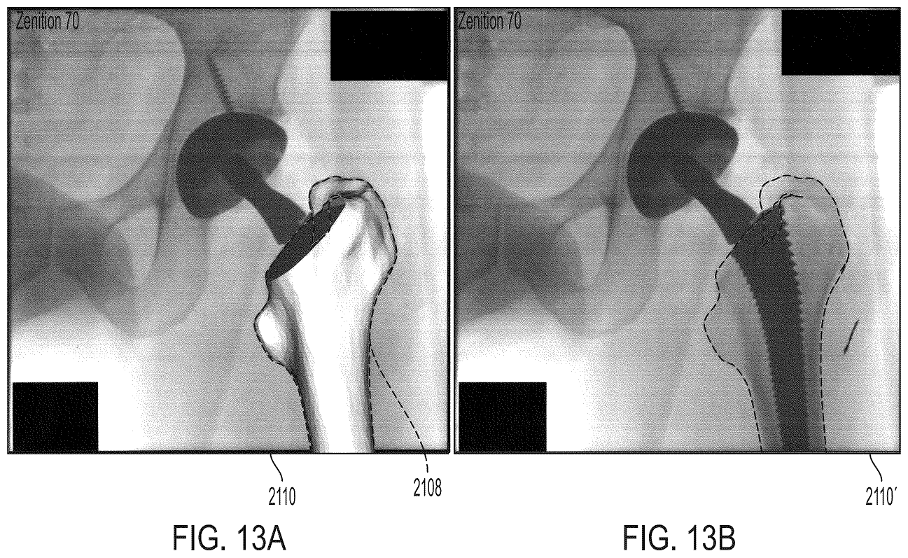

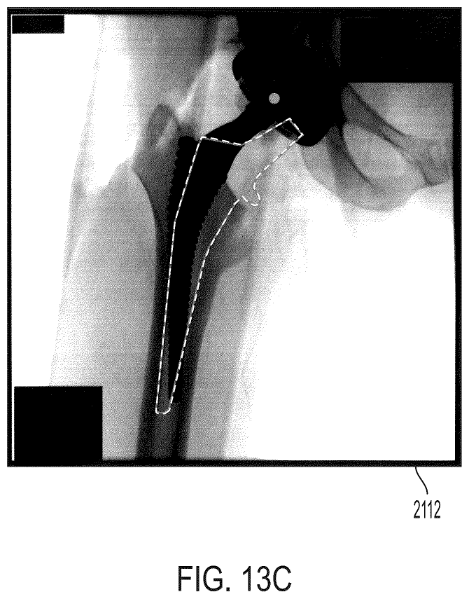

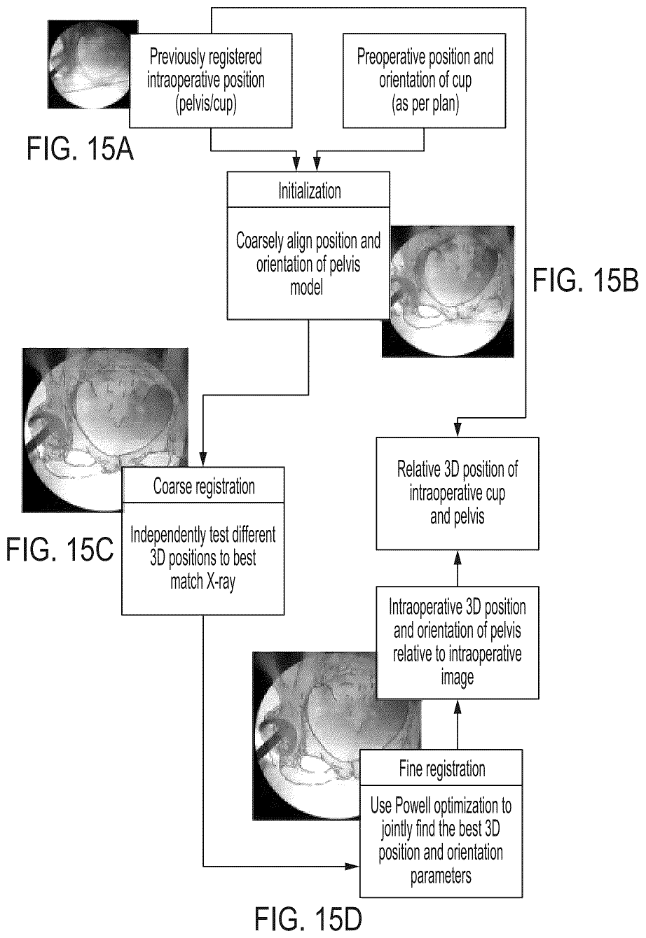

The system then matches a corresponding feature point(s) of the 3D bone model, for example the axis of the 3D bone model to the feature point on the achieved implantation image. FIG. 13A illustrates an exemplary initialization of a femur bone model 2108 (see also FIG. 15B illustrating an exemplary initialization of a pelvis). The feature point of bone surface(s) of the femur are used to match the bone surface of the femur bone model to the corresponding bone surface(s) of the femur in the achieved implantation image 2110. This provides an initial match position of the 3D bone model to the corresponding bone on the achieved implantation image. FIG. 13B is an exemplary achieved implantation image 2110′ illustrating the bone surface of a femur bone model silhouette overlayed with the intraoperative image of the patient's femur. FIG. 13C is another exemplary achieved implantation image 2112 illustrating an overlay of planned femur stem with an intraoperative image of a patient's femur.

While the foregoing initialization can be accomplished with a single feature point detection, the initial match position can be determined e.g., by aligning a plurality of parameters between the 3D bone model and the corresponding bone of the achieved implantation image. Such parameters can include, but not limited to, position (such as orientation), any major or anatomical feature of the bone, and/or a center, an axis, an edge, or surface of the bone.

Alternatively, the 3D bone model position can also be initialized using the known implant position from the implant registration phase and a planned relative alignment between bone and implant (from the surgical plan). This step can be performed without requiring any feature points defined on the achieved implantation image or bone.

Alternatively or in addition to, the bone initialization can be conducted by using or applying a registration from a prior image as the initialization for a current image. That is, the bone initialization can be achieved by positioning the 3D model according to the positioning parameters obtained from an earlier registration, e.g., from a registration that was performed on a preceding image in the surgical workflow. This allows to transfer positioning information between successively acquired 2D achieved implantation images.

In accordance with an alternative aspect of the subject disclosure, the preoperative data can include a selection of initial positions (including orientation(s)) and/or feature points of a subject bone based on the subject surgical procedure.

After initialization of the bone, the assessment moves to the registration phase of the 3D bone model to the bone in the achieved implantation image to determine a best fit of the 3D bone model to the bone in the achieved implantation image. The registration phase includes a coarse registration phase and a fine registration phase.

Referring to FIG. 10, the implant positioning system performs a first registration phase (also referred to as “coarse registration” or “coarse optimization”), applicable to the bone registration. During coarse registration of the bone, the system 100 independently tests difference values for each position parameter to best match the bone position as depicted in the achieved implantation image. This is accomplished by the system iteratively determining or generating a 2D projection image 2106a of the 3D bone model 2106 and assessing the position parameters of the 2D projection image compared to the position parameters of the bone in the intraoperative 2D image. The assessment includes determining a best match score based on the 2D information depicted in the 2D projection image compared to the information depicted in the intraoperative 2D image or achieved implantation image. The best match score is a predetermined score or an ideal score for determining when the iterative process of the coarse registration is considered to be completed.

The 2D projection images are generated by projecting the 3D model information and/or associated 3D points and features (such as 3D landmarks) into the 2D plane, given the position parameters of the model. The 2D projection images can depict the 2D contours or silhouettes of the 3D model, 2D landmark points, as well as other features of the model.

Additionally, the projection images can mimic clinical 2D X-ray or fluoroscopy images given the position parameters of the model. These “virtual X-rays” are computed by tracing rays through the model and accumulating the X-ray attenuation that is encountered by each ray passing through the mode (see e.g., FIG. 11, image of pelvis 2106a).

The match score or best match score is defined via a similarity in appearance of the bone model depicted in the 2D projection image and the bone depicted in the intraoperative image 2106c. The match score is computed based on the distance of corresponding features (e.g. silhouettes, landmarks) between the 2D projection image and the intraoperative image, and/or by comparing pixel intensity values in the 2D projection to pixel intensity values in the achieved implantation image through measures known in the art, such as mutual information or gradient correlation.

The position parameters can include feature points as discussed above and/or relative position and orientation, depth position, axis of the bone, angles covering six degrees of freedom, bony landmarks, offset, size and the like.

This process defines a coarse registration cycle because each position parameter can be modified in discrete increments e.g., increments of 5 degrees for angles, sizes in increments of 10% of overall size, and relative position in 1 mm increments. Alternatively, the discrete increments can be more or less, such as 4 or 6 degrees, sizes in increments of 5% or 15% or, relative position in 0.5 mm or 2 mm increments and the like. The iterative process ends when each parameter has been evaluated and an optimized best match score or overall best match score is achieved.

Upon completion of the coarse registration, the registration phase moves to the second registration phase (sometimes referred to as “fine registration” or “fine optimization”) (FIG. 10). During the second registration phase, the system refines the positions of the 3D computer model of the subject bone relative to the bone in the achieved implantation image. That is, the system refines the position determined during the first phase as the best match position. The best match position determined during the first phase is used as the initial position for the second registration phase, which then determines a best overall match or score for aligning the 3D bone model to the bone in the achieved implantation image. The best overall match or score is a predetermined score, or a score determined by the system based on data from the multi-parameter optimization, as further discussed below.

The second registration phase performs similar operations as the steps in the first phase but does so using multi-parameter optimization which performs a continuous search across all position parameters concurrently until a best fit position is determined for all parameters evaluated. That is, the system uses multi-parameter optimization to identify a best fit among one or more of the position parameters. The system can use e.g., Powell optimization (sometimes referred to as Powell's method or Powell's conjugate direction method) to identify a local minimum among the multitude of position parameters scored for determining a best fit position. Alternatively, other multi-parameter optimization techniques know in the art or to be developed and applicable to the present disclosure can also be used to optimize the second registration phase and determine the best fit position. Upon completion of the second registration phase, the 3D bone model is a registered 3D bone model.

### System Outputs

Once the computer-assisted implant positioning system completes the implant and bone registrations, the computer assesses the position of the registered 3D implant model relative to the registered 3D bone model defining an “achieved implant position” and compares the achieved implant position relative to the surgical plan, which can include a preoperative planned position of the subject implant in the patient and/or the planned preoperative position of the target bone. That is, the computer includes computer executable instructions to determine the position of the registered 3D implant model relative to the registered 3D bone model based on the subject surgical procedure and compares them to the surgical plan. For example, the achieved implant position can include a hip cup implant position relative to a patient's pelvis, a femur stem implant position relative to a patient's femur, femur bone position relative to the pelvis bone of a patient (to assess leg length and/or offset), or femur bone position relative to hip cup implant (to assess femur position relative to pelvis-acetabular construct) based on the femur stem implant position.

The assessment can include comparisons with respect to the overall alignment, relative position, orientation (including offset positions) or any other parameter related to the position/orientation, including e.g., inclination (such as inclination angles of an implant cup relative to a pelvis), anterior/posterior shift, medialization or superiolisation, anteversion, lengthening (such as lengthening of the leg), offset, version/extension/flexion, sagittal alignment, coronal alignment, area and volume, of the registered 3D computer model of the subject implant relative to registered 3D bone model, and changes in value to said parameters related to position/orientation. This assessment is collectively and individually referred to as “assessment data”.

The computer-assisted implant positioning system 100 displays, on a display operatively coupled to the computer, the assessment data 2002, as shown e.g., in FIG. 12. Based on the assessment data, the system is configured to determine and output recommended adjustments in the positioning of the surgical implants. Further, based on the assessment data, the system can output a visual of the registered 3D models on an intraoperative image 2000, with overlays (FIG. 13) or side-by-side comparisons (FIG. 12) to the patent-specific preoperative data.

For example, the system can display a visual indication of the determined real-time position of the registered 3D computer model of the subject implant and the registered 3D bone model relative to (e.g., superimposed on or side by side) the planned position of the implant in the patient or the planned preoperative position of the targe bone via an image 2004, including surgical plan data representative of an amount of deviation from the surgical plan 2002, 2006. Alternatively, the system can display the intraoperative patient-specific 2D image of the target bone having the implanted surgical implant (i.e., the achieved implantation image), relative to the planned position of the implant in the patient or the planned preoperative position of the targe bone via an image 2004, including surgical plan data representative of an amount of deviation from the surgical plan 2002, 2006. Exemplary data representative of an amount of deviation from the surgical plan can include, but not limited to implant orientation (e.g., inclination, and anteversion), bone discrepancy (e.g., leg length discrepancy), and implant offset (e.g., femoral offset), confirmation of implant and bone alignment, measurements of implant position across multiple points of anatomy, and comparison to preoperative positions and/or contralateral side of patient. Additionally, overlay of the 3D bone model to the initial or preoperative 2D image of the bone and overlay of the 3D computer model of the subject implant to the initial or preoperative 2D image of the subject implant allows the user to assess how well the implantation meets the surgical plan. Additionally, overlay of the achieved or planned implant model with the preoperative images or 2D intraoperative images allows the user to assess how well implantation meets the surgical plan, see e.g., FIG. 13C.

The various exemplary embodiments of the computer-assisted implant positioning system and method discussed herein provide numerous advantages over conventional implant positioning systems and methods. For example, the implant positioning system provides a user with real-time assessment data intraoperatively of how a surgical implant has been seated or implanted in a patient before the implant is permanently implanted, as well as a qualitative and quantitative assessment of how well the surgical implant position is matched with an ideal or planned surgical position for said implant. This allows a user to make adjustments in the positioning of a surgical implant intraoperatively.

The computer-assisted implant positioning system and method also advantageously provides for the ability to register two bodies in the same intraoperative image and relate their positions in three dimensions. The two bodies can an anatomical body (e.g., a bone of a patient) and a foreign body (e.g., a medical or surgical implant or instrument), but can alternatively be two anatomical bodies (e.g., two bones of a patient). In this manner the computer-assisted implant positioning system provides the ability to derive relative positions of e.g., two bones of a patient, such as a pelvis and femur in an image without registering any surgical implants or foreign bodies. Additionally, the computer-assisted implant positioning system can provide the relative positions of two bodies with images obtained prior implantation of any implants or before any bone cuts of any bones associated with the surgical procedure.

Additionally, the present disclosure provides a system for assessing the position of an implant intraoperatively based on 3D models obtained from 2D images. That is, the system utilizes 3D models to fully assess the 3D position of a surgical implant in a patient's bone intraoperatively based of a 2D patient-specific image obtained intraoperatively independent of or regardless of the orientation of the acquired 2D patient-specific image obtained intraoperatively. In other words, the system allows for determining the relative positions of the implants and bones, or any two bodies in three dimensions e.g., using 3D models. Additionally, the assessment performed by the system for assessing the intraoperative implant placement in the patient's bone does not rely upon or require any adjustment factor or correlating factor to account for patient pose relative to imaging equipment.

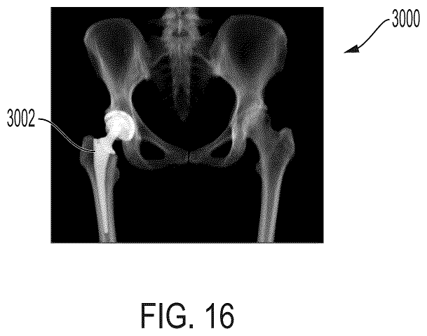

In accordance with another exemplary embodiment, the subject disclosure provides a computer-assisted implant positioning system substantially the same as computer-assisted implant positioning system 100 described above. However, instead of the processor receiving an intraoperative patient-specific image of the target bone associated with the surgical implant, the system receives a postoperative image (FIG. 16) of the target bone 3000 and implanted implant 3002. The system's processor is configured to then assess the postoperative image of the target bone and implanted implant relative to preoperative patient-specific data, such as the planned position of the subject implant in the target bone. Additionally, the user can assess the postoperative image of the target bone and implanted implant relative to the achieved implant position determined by the system based on the registered 3D computer model of the subject implant and the registered 3D bone model of the target bone. The system can then output the assessment of the postoperative image of the target bone and implanted implant on a display. This assessment based on postoperative image(s) can be used to determine postoperative care, rehabilitation and/or follow up treatment for the patient.

In accordance with another exemplary aspect of the subject disclosure, a computer-assisted implant positioning system is provided. The computer-assisted implant positioning system comprises: a receiver; a memory operatively connected to the receiver; and a processor operatively coupled to the receiver and the memory, the processor configured to: receive preoperative patient-specific data for a patient that includes: a 3D bone model of a target bone of the patient, and a planned position of a subject implant in the target bone, receive a 3D computer model of the subject implant, receive a postoperative patient-specific 2D image of the target bone associated with the surgical implant, register the 3D computer model of the subject implant to the postoperative patient-specific 2D image associated with the surgical implant and determine a best fit for the 3D computer model of the subject implant to the postoperative patient-specific 2D image associated with the surgical implant, register the 3D bone model of the target bone to the postoperative patient-specific 2D image of the target bone and determine a best fit for the 3D bone model to the postoperative patient-specific 2D image, determine an achieved implant position based on the registered 3D computer model of the subject implant and the registered 3D bone model of the target bone, and assess the postoperative patient-specific image relative to the preoperative patient-specific data, such as the planned position of the subject implant in the target bone.

In registering the 3D computer model of the subject implant to the postoperative patient-specific 2D image associated with the surgical implant to determine a best fit for the 3D computer model to the postoperative patient-specific 2D image, the processor is further configured to: acquire an initial position of the 3D computer model of the subject implant; assess the initial position of the 3D computer model of the subject implant to the postoperative patient-specific 2D image associated with the surgical implant; and refine the initial position of the 3D computer model of the subject implant to match the position of the surgical implant in the postoperative patient-specific 2D image.

In refining the initial position of the 3D computer model of the subject implant to match the position of the surgical implant in the postoperative patient-specific 2D image, the processor is further configured to match position based on one or more parameters selected from the group consisting of orientation, corners, depth, width, height, size, degree of freedom, and implant anatomical features.

In registering the 3D computer model of the subject implant to the postoperative patient-specific 2D image associated with the surgical implant to determine a best fit for the 3D computer model to the postoperative patient-specific 2D image, the processor is further configured to: determine the best fit using multi-parameter optimization based on a plurality of position parameters.

In registering the 3D bone model of the target bone to the postoperative patient-specific 2D image of the target bone to determine a best fit for the 3D bone model to the postoperative patient-specific 2D image, the processor is further configured to: acquire an initial position of the 3D bone model of the target bone; assess the initial position of the 3D bone model of the target bone to the postoperative patient-specific 2D image of the target bone; and refine the initial position of the 3D bone model of the target bone to match the position of the target bone in the postoperative patient-specific 2D image.

In accordance with yet another exemplary embodiment, the subject disclosure provides an implant positioning method comprising: acquiring, using a patient imaging device, a postoperative patient-specific image of a target bone associated with a surgical implant; acquiring, using a computer, a 3D bone model of the target bone; acquiring, using the computer, a 3D computer model of a subject implant implanted in the target bone; registering, using the computer, a position of the 3D computer model of the subject implant relative to a position of the surgical implant in the postoperative image of the target bone; registering, using the computer, a position of the 3D bone model of the target bone to a position of the target bone in the postoperative image of the target bone; assessing, using the computer, the position of the registered 3D computer model of the subject implant relative to the position of the registered 3D bone model; evaluating, using the computer, the assessed position of the registered 3D computer model of the subject implant and target bone relative to a planned position of the surgical implant in the target bone of the patient; and outputting on a display, the evaluation of the assessed position of the registered 3D computer model of the subject implant and target bone relative to a planned position of the surgical implant in the target bone of the patient. The registering step of the 3D computer model of the subject implant comprises: determining, using the computer, an initial position of the 3D computer model of the subject implant relative to a position of the surgical implant in the target bone in the postoperative patient-specific image; and refining the initial position of the 3D computer model of the subject implant to match the position of the surgical implant in the target bone in the postoperative patient-specific 2D image.

The step of refining the initial position of the 3D computer model of the subject implant comprises determining a best fit by independently evaluating a plurality of parameters for matching the 3D computer model of the subject implant to the position of the surgical implant in the postoperative patient-specific 2D image. The step of refining the initial position of the 3D computer model of the subject implant comprises determining a best fit using multi-parameter optimization to optimize a plurality of parameters for matching the 3D computer model of the subject implant to the position of the surgical implant in the postoperative patient-specific 2D image.

The registering step of the 3D bone model of the target bone comprises: determining, using the computer, an initial position of the 3D bone model of the target bone relative to a position of the target bone in the postoperative patient-specific image; and refining the initial position of the 3D bone model of the target bone to match the position of the target bone in the postoperative patient-specific 2D image.

The step of refining the initial position of the 3D bone model of the target bone comprises determining a best fit by independently evaluating a plurality of parameters for matching the 3D bone model of the target bone to the position of the target bone in the postoperative patient-specific 2D image. The step of refining the initial position of the 3D bone model of the target bone comprises determining a best fit using multi-parameter optimization to optimize a plurality of parameters for matching the 3D bone model of the target bone to the position of the target bone in the postoperative patient-specific 2D image.

### EXAMPLES

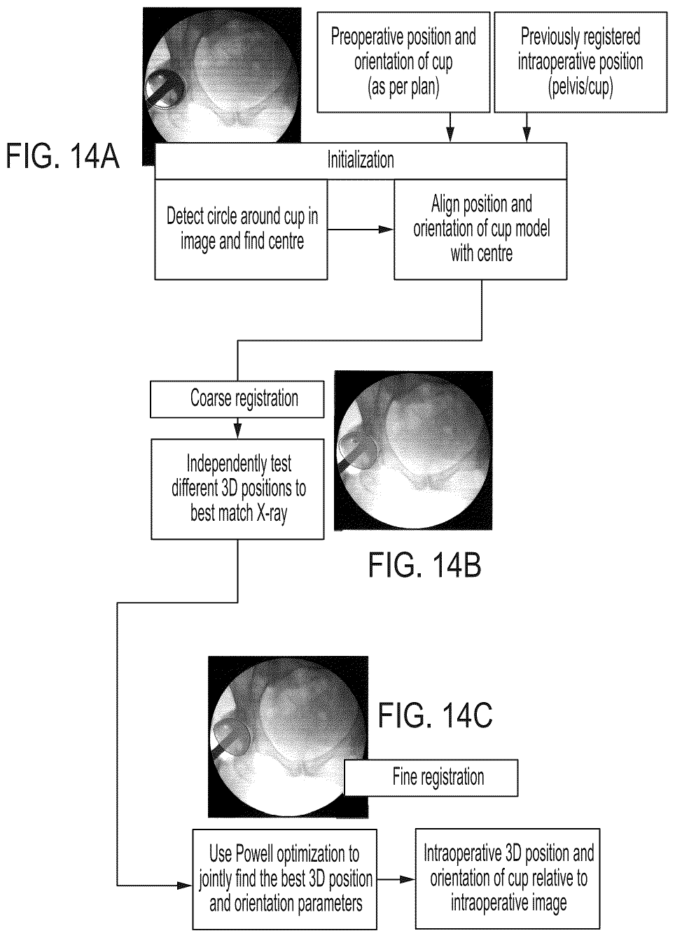

FIGS. 14A-C illustrate and exemplary example of hip cup implant registration. In this example, the surgical plan includes preoperative position and orientation data of the hip cup implant. Initialization was performed by detecting the circle around the hip cup implant in an intraoperative X-ray (e.g., a fluoroscopy image) image of the hip cup implant on the acetabulum and finding its center. Then the position and orientation of the hip cup implant was aligned with the center and orientation as per the surgical plan. Thereafter, coarse registration was performed by independently testing different 3D positions of the hip cup implant to best match the X-ray of the acetabulum. Afterwards, fine registration was performed using Powell optimization to jointly find the best 3D position and orientation parameters.

FIGS. 15A-D illustrate an exemplary example of a pelvis bone registration. In this example, the surgical plan includes preoperative position and orientation data of the pelvis relative to the cup implant. Additional inputs to this pelvis bone registration include the 3D position and orientation of the hip cup implant from the hip cup implant registration stage (FIG. 15A which illustrates the 3D position and orientation of the hip cup implant). Initialization was performed by aligning the position and orientation of the pelvis bone model with the registered hip implant cup model per the surgical plan (FIG.

Coarse registration was performed by independently testing different 3D positions to find a best match with the intraoperative X-ray of the pelvis (FIG. 15C). Thereafter, fine registration was performed using Powell optimization to jointly find the best 3D position and orientation parameters (FIG. 15D which illustrates intraoperative 3D position and orientation of the hip cup implant relative to intraoperative images of the pelvis and the intraoperative relative 3D position of the hip cup implant and pelvis).

It will be appreciated by those skilled in the art that changes could be made to the various aspects and exemplary embodiments described above without departing from the broad inventive concept thereof. It is to be understood, therefore, that the subject application is not limited to the particular aspects or exemplary embodiments disclosed, but it is intended to cover modifications within the spirit and scope of the subject application as defined by the appended claims.

We claim:

1. A computer-assisted implant positioning system comprising:
a receiver;
a memory operatively connected to the receiver; and
a processor operatively coupled to the receiver and the memory, the processor configured to:
receive preoperative patient-specific data for a patient that includes:
a 3D bone model of a target bone of the patient, and
a planned position of a subject implant in the target bone,
receive a 3D computer model of the subject implant,
receive an intraoperative patient-specific 2D image of the target bone associated with the surgical implant,
register the 3D computer model of the subject implant to the intraoperative patient-specific 2D image associated with the surgical implant and determine a best fit for the 3D computer model of the subject implant to the intraoperative patient-specific 2D image associated with the surgical implant,
register the 3D bone model of the target bone to the intraoperative patient-specific 2D image of the target bone and determine a best fit for the 3D bone model to the intraoperative patient-specific 2D image,
determine an achieved implant position based on the registered 3D computer model of the subject implant and the registered 3D bone model of the target bone, and
assess the achieved implant position relative to the preoperative patient-specific data.

2. The computer-assisted implant positioning system of claim 1, wherein the processor is further configured to assess the achieved implant position relative to the preoperative patient-specific data to obtain assessment data.

3. The computer-assisted implant positioning system of claim 2, wherein the assessment data includes a degree of freedom, orientations, an offset, a lengthening, or combinations thereof.

4. The computer-assisted implant positioning system of claim 2, wherein the processor is further configured to output on a display one or more of the assessment data.

5. The computer-assisted implant positioning system of claim 1, wherein in registering the 3D computer model of the subject implant to the intraoperative patient-specific 2D image associated with the surgical implant to determine a best fit for the 3D computer model to the intraoperative patient-specific 2D image, the processor is further configured to:
acquire an initial position of the 3D computer model of the subject implant;
assess the initial position of the 3D computer model of the subject implant to the intraoperative patient-specific 2D image associated with the surgical implant; and
refine the initial position of the 3D computer model of the subject implant to match the position of the surgical implant in the intraoperative patient-specific 2D image.

6. The computer-assisted implant positioning system of claim 5, wherein in refining the initial position of the 3D computer model of the subject implant to match the position of the surgical implant in the intraoperative patient-specific 2D image, the processor is further configured to match position based on one or more parameters selected from the group consisting of orientation, corners, depth, width, height, size, degree of freedom, and implant anatomical features.

7. The computer-assisted implant positioning system of claim 1, wherein in registering the 3D computer model of the subject implant to the intraoperative patient-specific 2D image associated with the surgical implant to determine a best fit for the 3D computer model to the intraoperative patient-specific 2D image, the processor is further configured to:
determine the best fit using multi-parameter optimization based on a plurality of position parameters.

8. The computer-assisted implant positioning system of claim 7, wherein the plurality of position parameters includes a degree of freedom, orientation, corners, a depth, a width, a height, a size, and/or implant anatomical features.

9. The computer-assisted implant positioning system of claim 1, wherein in registering the 3D bone model of the target bone to the intraoperative patient-specific 2D image of the target bone to determine a best fit for the 3D bone model to the intraoperative patient-specific 2D image, the processor is further configured to:
acquire an initial position of the 3D bone model of the target bone;
assess the initial position of the 3D bone model of the target bone to the intraoperative patient-specific 2D image of the target bone; and
refine the initial position of the 3D bone model of the target bone to match the position of the target bone in the intraoperative patient-specific 2D image.

10. The computer-assisted implant positioning system of claim 1, further comprising:
a patient imaging device operatively in communication with the receiver; and
a display operatively connected to the processor.

11. The computer-assisted implant positioning system of claim 1, wherein the preoperative patient-specific data includes a surgical plan.

12. The computer-assisted implant positioning system of claim 1, wherein the processor is further configured to receive a patient-specific image of a target bone of the patient.

13. The computer-assisted implant positioning system of claim 1, wherein the subject implant is a medical implant, a surgical instrument, an implant trial, or a foreign object.

14. The computer-assisted implant positioning system of claim 1, wherein the processor is further configured to determine the achieved implant position in 3D relative to a target bone.

15. An implant positioning method comprising:
acquiring, using a patient imaging device, an intraoperative patient-specific image of a target bone associated with a surgical implant;
acquiring, using a computer, a 3D bone model of the target bone;
acquiring, using the computer, a 3D computer model of a subject implant to be implanted in the target bone;
registering, using the computer, a position of the 3D computer model of the subject implant relative to a position of the surgical implant in the intraoperative image of the target bone;
registering, using the computer, a position of the 3D bone model of the target bone to a position of the target bone in the intraoperative image of the target bone;
assessing, using the computer, the position of the registered 3D computer model of the subject implant relative to the position of the registered 3D bone model;
evaluating, using the computer, the assessed position of the registered 3D computer model of the subject implant and target bone relative to a planned position of the surgical implant in the target bone of the patient; and
outputting on a display, the evaluation of the assessed position of the registered 3D computer model of the subject implant and target bone relative to a planned position of the surgical implant in the target bone of the patient.

16. The implant positioning method of claim 15, wherein the registering step of the 3D computer model of the subject implant comprises:
determining, using the computer, an initial position of the 3D computer model of the subject implant relative to a position of the surgical implant in the target bone in the intraoperative patient-specific image; and
refining the initial position of the 3D computer model of the subject implant to match the position of the surgical implant in the target bone in the intraoperative patient-specific 2D image.

17. The implant positioning method of claim 16, wherein the step of refining the initial position of the 3D computer model of the subject implant comprises determining a best fit by independently evaluating a plurality of parameters for matching the 3D computer model of the subject implant to the position of the surgical implant in the intraoperative patient-specific 2D image.

18. The implant positioning method of claim 16, wherein the step of refining the initial position of the 3D computer model of the subject implant comprises determining a best fit using multi-parameter optimization to optimize a plurality of parameters for matching the 3D computer model of the subject implant to the position of the surgical implant in the intraoperative patient-specific 2D image.

19. The implant positioning method of claim 15, wherein the registering step of the 3D bone model of the target bone comprises:
determining, using the computer, an initial position of the 3D bone model of the target bone relative to a position of the target bone in the intraoperative patient-specific image; and
refining the initial position of the 3D bone model of the target bone to match the position of the target bone in the intraoperative patient-specific 2D image.

20. The implant positioning method of claim 19, wherein the step of refining the initial position of the 3D bone model of the target bone comprises determining a best fit by independently evaluating a plurality of parameters for matching the 3D bone model of the target bone to the position of the target bone in the intraoperative patient-specific 2D image.

21. The implant positioning method of claim 19, wherein the step of refining the initial position of the 3D bone model of the target bone comprises determining a best fit using multi-parameter optimization to optimize a plurality of parameters for matching the 3D bone model of the target bone to the position of the target bone in the intraoperative patient-specific 2D image.

22. A computer-assisted implant positioning system comprising:
a receiver;
a memory operatively connected to the receiver; and
a processor operatively coupled to the receiver and the memory, the processor configured to:
receive preoperative patient-specific data for a patient that includes:
a 3D bone model of a target bone of the patient, and
a planned position of a subject implant in the target bone,
receive a 3D computer model of the subject implant,
receive a postoperative patient-specific 2D image of the target bone associated with the surgical implant,
register the 3D computer model of the subject implant to the postoperative patient-specific 2D image associated with the surgical implant and determine a best fit for the 3D computer model of the subject implant to the postoperative patient-specific 2D image associated with the surgical implant,
register the 3D bone model of the target bone to the postoperative patient-specific 2D image of the target bone and determine a best fit for the 3D bone model to the postoperative patient-specific 2D image,
determine an achieved implant position based on the registered 3D computer model of the subject implant and the registered 3D bone model of the target bone, and
assess the postoperative patient-specific image relative to the preoperative patient-specific data.
